(19) United States
(12) Patent Application Publication
(10) Pub. No.: US 2010/0092947 A1
(43) Pub. Date: Apr. 15, 2010

(54) Impdh2 snp associated with acute rejection

(76) Inventors: Laurent Essioux, Attenschwiller (FR); Dorothee Foernzler, Lenzburg (CH); Lara Hashimoto, North York, CA (US); Klaus Lindpaintner, Muttenz (CH); Michelle Rashford, Hertfordshire (GB); Olivia Spleiss, Freiburg (DE); Matt Truman, North Manly (AU); Athina Voulgari, London (GB)

Correspondence Address: HOFFMANN-LA ROCHE INC.;PATENT LAW DEPARTMENT, 340 KINGSLAND STREET, NUTLEY, NJ, 07110, US

(21) Appl. No.: 11/921,996

(22) PCT Filed: Jun. 7, 2006
(86) PCT No.: PCT/EP06/05440

§ 371 Date: Jul. 7, 2009

(30) Foreign Application Priority Data
Jun. 13, 2005 (EP) 05105163.9

(51) Int. Cl. C12Q 1/68

(52) U.S. Cl. 435/6

(57) ABSTRACT

The present invention concerns a method for the prediction of acute renal transplant rejection by detecting a polymorphism in intron 7 of the IMPDH2 gene, optionally in combination with polymorphisms of the MDR1 and IL 10 genes which were found to be associated with this disease.

The present invention relates to a marker for acute rejection of renal transplants.

Renal transplantation is frequently associated with acute rejection of the transplant. It would be advantageous to identify gene polymorphisms in immunoregulatory genes that would allow predicting the individual risk of poor post-transplant outcome, as well as predicting which patients are more susceptible to developing adverse side effects and/or which patients are likely to progress to more severe disease states and renal failure. Additionally, since the genetic variation in the target or in the biosynthetic and metabolic pathway of a drug may influence the individual's response to therapy, gene polymorphisms associated with mycophenolate mofetil (MMF), or Cyclosporin A (CsA) metabolism would be useful as markers to predict response to therapy.

The present invention is based on the association of a single nucleotide polymorphism (SNP) in the IMPDH2 (inosine monophosphate dehydrogenase 2) gene locus (T3757C) with rejection of renal transplants. This gene locus corresponds to position 3757 of Seq ID No.1. Seq ID No. 1 represents exons 1 to 13 of the IMPDH2 gene. The polymorphism at this position consists of the replacement of the nucleotide T by a C in intron 7 of the IMPDH2 gene locus.

IMPDH2 is the rate-limiting enzyme for de novo GMP synthesis in activated lymphocytes. Its activity is correlated with cell growth, and it is the target of a number of proven and experimental drug therapies including MMF. MPA is the active metabolite to which MMF, the 2-morpholinoethyl ester of MPA, hydrolyses to, following oral administration of the drug; MPA potently, selectively and reversibly inhibits IMPDH2. Changes in expression of IMPDH2 have been associated with transplant rejection (Vannozzi et al., Transplant Proc. 2004, 36(9), 2787-2790).

The present invention relates to a previously unknown polymorphism of the IMPDH gene which is shown herein to be associated with acute renal transplant rejection. Said polymorphism is located at the nucleotide at position 3757 of Seq ID No. 1, wherein the T at this position is replaced by a C. Isolated nucleic acid molecules comprising Seq ID No. 1 and fragments thereof wherein the nucleotide T at position 3757 of Seq ID No. 1 is replaced by a C are, thus, indicative for the susceptibility of a patient to acute renal transplant rejection. Thus, in one embodiment, the present invention relates to an isolated nucleic acid molecule comprising Seq ID No. 1, wherein the nucleotide T at position 3757 is replaced by a C. In another embodiment, the present invention also relates to an isolated nucleic acid molecule comprising a fragment of Seq ID No. 1, which includes position 3757 of Seq ID No. 1 and wherein nucleotide T at position 3757 is replaced by a C.

The present invention further relates to a method for assessing the susceptibility to acute renal transplant rejection in a patient, comprising a) isolating a nucleic acid from a sample that has been removed from the patient, and b) detecting the nucleotide present at position 3757 of SEQ ID No. 1, wherein the presence of a C at this position is indicative of renal transplant rejection, c) optionally detecting one or more other marker for the prediction of acute renal transplant rejection.

Said optional one or more markers may be polymorphisms present in other genes. Preferably, step c) of the above method comprises detecting the nucleotide present at position 176 of Seq. ID No. 5 (C3435T), wherein the presence of a T at this position is indicative of renal transplant rejection, and/or the detection of the nucleotide at position 682 of Seq ID No. 6 (−592 C>A), wherein the presence of an A at this position is indicative of renal transplant rejection. Seq ID No. 5 refers to exon 26 of the human multidrug resistance 1 (MDR1) gene. Seq ID No. 6 refers to the promoter region of the human Interleukin 10 (IL 10) gene.

Preferably, the sample which is used for the above method is whole blood. Said sample may also be serum or plasma.

Detection of the nucleotides hereinbefore described can be performed by any method which is suitable for genotyping. The presence of the polymorphism in the IMPDH nucleic acid sequence described above can be determined by a differential nucleic acid analysis technique such as restriction fragment length polymorphism analysis, direct mass-analysis of PCR products using mass spectrometry, direct analysis of invasive cleavage products, extension-based techniques such as ARMS™ (amplification refractory mutation system), ALEX™ (amplification refractory mutation system linear extension) and COPS (competitive oligonucleotide priming system), OLA (oligonucleotides ligation assay), Invader assay, direct sequence analysis or polymerase chain reaction analysis.

In one preferred embodiment, the method is dideoxy sequencing. One more preferred method is thermocycle sequencing.

For the detection of the IMPDH polymorphism described above, in a most preferred embodiment, said sequencing is performed using the primers of Seq ID No. 10 and Seq ID No. 11.

A preferred method of detecting the nucleotides of any one of the polymorphisms is quantitative PCR.

In a more preferred embodiment, said method is quantitative allele-specific PCR using allele-specific primers that only anneal to one allele and not to the other. One such quantitative PCR is kinetic thermal cycling (KTC). One more preferred detection method is amplification refractory mutation systems (ARMS) together with KTC, which allows discrimination of single nucleotide polymorphisms (SNP) in a single-tube without the use of fluorescent probes (Higuchi et al., Biotechnology (1993), 11, 1026-1030).

For the additional optional detection of the IL10 polymorphism with the method described above, in a most preferred embodiment, said allele-specific PCR is performed using the allele-specific primers of Seq ID No. 2 and Seq ID No. 3 and the common primer of Seq ID No. 4.

For the additional optional detection of the MDR1 polymorphism described above, a most preferred embodiment comprises performing allele-specific PCR using the allele-specific primers of Seq ID No. 7 and Seq ID No. 8 and the common primer of Seq ID No. 9.

In a most preferred embodiment, the additional detection in step c) comprises detection of both the MDR1 and IL 10 polymorphisms.

The additional optional detection in step c) of the method hereinbefore described may be performed in the same or in separate reaction mixtures.

Samples can be tested for the presence of nucleic acid sequences which are different from normal sequences using any one of a wide variety of differential nucleic acid analysis techniques that are well known in the art. Differential nucleic acid analysis techniques include, but are not limited to: fluorescent in situ hybridization (FISH), direct DNA sequencing, single stranded conformational analysis (SSCP), Southern blotting including restriction fragment length polymorphism analysis (RFLP), the polymerase chain reaction (PCR), polymorphism specific oligonucleotide hybridizations and PCR-SSCP analysis, single nucleotide primer extension and oligonucleotides ligation. For a review of techniques for evaluating and manipulating nucleic and amino acid sequences, see Current Protocols In Molecular Biology, Volumes I-III, Frederick M. Ausubel et al. eds., 1995. Hybridization methods include, but are not limited to, Reverse dot blot, GeneChip microarrays, DASH, PNA and LNA probes, TaqMan and Molecular Beacons; allele-specific PCR includes, but is not limited to, Intercalating dye, FRET primers and ALphaScreen; primer extension includes, but is not limited to, SNPstream/GBA (genetic bit analysis), multiplex minisequencing/SNaPshot, Pyrosequencing, MassEXTEND/MassArray, GOOD assay, Microarray miniseq/APEX (arrayed primer extension), microarray primer extension, ‘Tag’ arrays, coded microspheres, TDI (template-directed incorporation)/fluorescence polarization; oligonucleotide ligation includes, but is not limited to, colorimetric OLA (oligonucleotide ligation assay), sequence-coded OLA, microarray ligation, ligase chain reaction, padlock probes, rolling circle amplification; endonuclease cleavage includes, but is not limited to Restriction site analysis and Invader assay. These methods are described in Syvänen, Nature Rev Genet 2, 2001, 930-942, and the references cited therein. Multiplex platforms for high marker complexity include, but are not limited to, bead-based multiplex genotyping, high density microarrays for genotyping, re-sequencing platforms, microarray-based analysis of differential gene expression. These methods are described in Koch, Nature Rev Drug Discovery 3, 2004, 749-761 and the references cited therein. Combinations of the methods listed above can also be used (e.g. as a non-limiting example, a combination of molecular inversion probes and hybridization on arrays, Hardenbol et al., Nat. Biotechnol. 2003, 21, 673-678.

A number of methods can be used to directly detect DNA sequence variation. Direct DNA sequencing, either manual sequencing or automated fluorescent sequencing can detect sequence variation. Besides dideoxy sequencing, pyrosequencing can also be used. The allele(s) of genes in the IMPDH2 region in an individual to be tested can be cloned using conventional techniques. For example, a blood sample is obtained from the individual, IMPDH2 genomic DNA is isolated from the cells in this sample and ligated into an appropriate vector for amplification. The sequences of the clones can then be determined and compared to the normal IMPDH2 sequences. Techniques involving DNA cloning and sequencing are well known in the art, see e.g. Current Protocols In Molecular Biology, Volume 1, unit 7, Frederick M. Ausubul et al. eds., 1995.

Another approach to detect variations in DNA sequences is the single-stranded conformation polymorphism assay (SSCP) (Orita et al., 1989). This method does not detect all sequence changes, especially if the DNA fragment size is greater than 200 bp, but can be optimized to detect most DNA sequence variation. The reduced detection sensitivity is a disadvantage, but the increased throughput possible with SSCP makes it an attractive, viable alternative to direct sequencing for polymorphism detection on a research basis. The fragments which have shifted mobility on SSCP gels are sequenced to determine the exact nature of the DNA sequence variation. Other approaches based on the detection of mismatches between the two complementary DNA strands include clamped denaturing gel electrophoresis (CDGE) (Sheffield et al., Am. J. Hum. Genet., 49: 699-706 (1991)), heteroduplex analysis (NA) Mite et al., Genomics 12: 301-306 (19921) and chemical mismatch cleavage (CMC) (Grompe et al., P.N.A.S. 86: 5855-5892 (1989)). Other methods which might detect these classes of polymorphisms such as a protein truncation assay or the asymmetric assay detect only specific types of polymorphisms and would not detect missense polymorphisms. A review of currently available methods of detecting DNA sequence variation can be found in a recent review by Grompe et al., Nature Genetics 5: 111-117, (1993) and Landegren et al, Genome Research 8:769-776, (1998).

A rapid preliminary analysis to detect polymorphisms in DNA sequences can be performed using RFLP, where DNA is cut with one or more restriction enzymes, preferably with a large number of restriction enzymes and analyzed with IMPDH2 specific probes in a series of Southern blots. Each blot contains a series of normal individuals and a series of cases with abnormal bone formation. Southern blots displaying hybridizing fragments (differing in length from control DNA when probed with sequences near or including known polymorphic loci) indicate a possible polymorphism. Techniques involving RFLP are well known in the art, see, e.g., Current Protocols In Molecular Biology, Volume 1, unit 2, Frederick M. Ausubul et al. eds., 1995.

Restriction fragment length polymorphism analysis is a preferred method of analysis due to its ability to identify uncharacterized polymorphisms. Specifically, by simply using sequences from various regions in IMPDH2 as probes, the skilled practitioner may evaluate nucleic acid samples for the IMPDH2 T3757C polymorphism disclosed herein or alternatively, may include proximal sequences identified herein or isolated by chromosomal walking techniques that are well known in the art. See e.g. Ueghara et al., Mamm. Genome 1(2): 92-99 (1991).

A particularly preferred method of nucleic acid analysis using polymerase-driven amplification is the polymerase chain reaction (PCR). The polymerase chain reaction and other polymerase-driven amplification assays can achieve over a million-fold increase in copy number through the use of polymerase-driven amplification cycles. Once amplified, the resulting nucleic acid can be analyzed by restriction endonuclease digestion, sequenced or used as a substrate for DNA probes. When the sequences encompassing a specific polymorphism are known, a variety of PCR primers targeting these sequences may be generated. For example, sequences flanking the polymorphism may be used to amplify those sequences. For a variation of sequence-specific PCR, primers can be used which hybridize at their 3′ ends to the IMPDH2 T3757C polymorphism. If the particular polymorphism is not present, an amplification product is not observed. Amplification Refractory Polymorphism System (ARMS) can also be used, as disclosed in European Patent Application Publication No. 0332435 and in Newton et al., 1989. PCRs can also be performed with primer pairs based on any sequence of the normal IMPDH2 region. For example, primer pairs for one of the introns can be prepared and utilized. The amplified products are then analyzed by single stranded conformation polymorphisms (SSCP) using conventional techniques to identify any differences and these are then sequenced and compared to the normal gene sequence.

Other suitable amplification methods include the ligase chain reaction (LCR) (see Wu and Wallace, Genomics 4:560 (1989), Landegren et al., Science 241:1077 (1988), transcription amplification (Kwoh et al., Proc. Natl. Acad. Sci. USA 86:1173 (1989)), and self-sustained sequence replication (Guatelli et al., Proc. Nat. Acad. Sci. USA 87:1874 (1990)) and nucleic acid based sequence amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively.

Primer pairs of the present invention are useful for determination of the nucleotide sequence of a particular IMPDH2 sequence using PCR. For example, the pairs of single-stranded DNA primers can be annealed to sequences within or surrounding IMPDH2 sequences in order to prime amplifying DNA synthesis of the gene itself. A complete set of these primers allows synthesis of all of the nucleotides of the gene coding sequences. The set of primers preferably allows synthesis of both intron and exon sequences. In addition, allele-specific primers can also be used. Such primers anneal only to the IMPDH2 alleles wherein T3757 is replaced by a C, and thus will only amplify a product in the presence of the mutant allele as a template. DNA sequences of the IMPDH2 region which have been amplified by use of PCR may also be screened using allele-specific probes. These probes are nucleic acid oligomers, each of which contains a region of the gene sequence harbouring the T3757C polymorphism. For example, one oligomer may be about 20 nucleotides in length, corresponding to a portion of the IMPDH2 polymorphic sequence. Hybridization of allele-specific probes with amplified IMPDH2 sequences can be performed, for example, on a nylon filter. Hybridization to a particular probe under stringent hybridization conditions indicates the presence of the same polymorphism in the tissue as in the allele-specific probe.

Another preferentially applied method to detect polymorphisms includes the use of mass spectrometry. After the PCR amplification of a DNA sequence that contains the IMPDH T3757C polymorphism, an internal primer extension reaction is carried out with a primer ending one base upstream from the polymorphism of interest. Using only dideoxynucleoside triphosphates (ddNTPs) in the primer extension reaction, the primer will be extended by only one base which represents the polymorphic. The exact mass of the extended primer is determined directly with MALDI-TOF (Matrix Assisted Laser Desorption Ionization—Time of Flight) mass spectrometry and heterozygotes generate 2 peaks that can be unambiguously distinguished.

Invasive cleavage products may also be detected by mass spectrometry or by fluorescent based methods. Single nucleotide polymorphisms (SNPs) are detected based on the ability of special structure-specific endonucleases (cleavases) to recognize specific DNA structures (created by a specific hybridization). An invader probe and a labeled signal probe are designed to hybridize to the target DNA so that the Invader probe overlaps the signal probe by at least one base representing the SNP site. This invasion of the signal-probe target duplex displaces a single-stranded flap containing the label. The juncture between the flap and the partially invaded duplex is recognized and cleaved by the enzyme only in case of complementary bases at the cleavage site, releasing the unhybridized region of the signal probe. Detection of the cleaved fragment can be accomplished as described above or by direct gel analysis or enzyme-linked antibody to a tag on the fragment. After cleavage, a new signal probe hybridizes and the process repeats, so that the cleaved signal probe accumulates. The signal is therefore amplified in this method and this amplification increases the overall sensitivity of the technique.

Vice versa, several polymorphism-containing oligonucleotides may be immobilized on a nylon filter (“SNP strip”) and hybridized with the products of a multiplex PCR reaction obtained from the DNA of an individual for allele-specific hybridisation (Cheng et al., Clin. Chem. Lab. Med. (1998) 36(8): 561-566, RMS, Alameda).

The majority of the assays described above incorporate nucleic acid probes as a crucial element. When the probes are used to detect the presence of the target sequences, the biological sample to be analyzed, such as blood or serum, may be treated to extract the nucleic acids. As discussed above, the sample nucleic acid may be prepared in various ways to facilitate detection of the target sequence, e.g., denaturation, restriction digestion, electrophoresis or dot blotting. The targeted region of the analyte nucleic acid usually must be at least partially single-stranded to form hybrids with the targeting sequence of the probe. If the sequence is naturally single-stranded, denaturation will not be required. However, if the sequence is double-stranded, the sequence will probably need to be denatured. Denaturation can be carried out by various techniques known in the art.

Target nucleic acids, probe and analyte can be incubated under conditions which promote stable hybrid formation of the target sequence in the probe with the putative targeted sequence in the analyte. The region of the probe which is used to bind to the analyte can be made completely complementary to the targeted region of the human IMPDH2 gene. Therefore, high stringency conditions are desirable in order to prevent false positives. However, conditions of high stringency are used only if the probes are complementary to regions of the chromosome which are unique in the genome. The stringency of hybridization is determined by a number of factors during hybridization and during the washing procedure, including temperature, ionic strength, base composition, probe length, and concentration of formamide. These factors are outlined in, for example, Maniatis et al., Molecular Cloning, A Laboratory Manual, Cold Springs Harbor Laboratory, 1982 and Sambrook et al., 1989. Under certain circumstances, the formation of higher order hybrids, such as triplexes, quadraplexes, etc., may be desired to provide the means of detecting target sequences.

Nucleic acid hybridization will be affected by such conditions as salt concentration, temperature, or organic solvents, in addition to the base composition, length of the complementary strands, and the number of nucleotide base mismatches between the hybridizing nucleic acids, as will be readily appreciated by those skilled in the art. Stringent temperature conditions will generally include temperatures in excess of 30° C., typically in excess of 37° C., and preferably in excess of 45° C. Stringent salt conditions will ordinarily be less than 1000 mM, typically less than 500 mM, and preferably less than 200 mM. However, the combination of parameters is much more important than the measure of any single parameter. Probe sequences may also hybridize specifically to duplex DNA under certain conditions to form triplex or other higher order. DNA complexes. The preparation of such probes and suitable hybridization conditions are well known in the art.

Detection, if any, of the resulting hybrid is usually accomplished by the use of labeled probes. Alternatively, the probe may be unlabeled, but may be detectable by specific binding with a ligand which is labeled, either directly or indirectly. Suitable labels, and methods for labeling probes and ligands are known in the art, and include, for example, radioactive labels which may be incorporated by known methods (e.g., nick translation, random priming or kinasing), biotin, fluorescent groups, chemiluminescent groups (e.g., dioxetanes, particularly triggered dioxetanes), enzymes, antibodies and the like. Variations of this basic scheme are known in the art, and include those variations that facilitate separation of the hybrids to be detected from extraneous materials and/or that amplify the signal front the labeled moiety. A number of these variations are reviewed in, e.g., Matthews & Kricka, Anal. Biochem., 169: 1, 1988; Landegren et al., Science, 242; 229, 1988; Mittlin, 1989; U.S. Pat. No. 4,868,105; and in EPO Publication No. 225,807.

Nucleic acid sequences having polymorphisms associated with acute renal transplant rejection can be detected by hybridization with a polynucleotide probe which forms a stable hybrid with that of the target sequence, under stringent to moderately stringent hybridization and wash conditions. The present invention allows for the design of probes which preferentially hybridize to polymorphic regions. The design of probes which preferentially target specific sequences and hybridization conditions for their use is well known in the art. See e.g. Current Protocols In Molecular Biology, Volumes I-III, Frederick M. Ausubel et al. eds., 1995. For example, if it is expected that the probes will be perfectly complementary to the target sequence, stringent conditions will be used. Hybridization stringency may be lessened if some mismatching is expected, for example, if variants are expected with the result that the probe will not be completely complementary. Conditions are chosen which rule out nonspecific/adventitious bindings in order to minimize noise.

The probes can include an isolated polynucleotide attached to a label or reporter molecule and may be used to isolate other polynucleotide sequences, having sequence similarity or being proximal to the sequences of interest by standard methods. For techniques for preparing and labeling probes see, e.g., Sambrook et al., 1989 or Ausubel et al., 1992. Other similar polynucleotides may be selected by using homologous polynucleotides. Probes comprising synthetic oligonucleotides or other polynucleotides of the present invention may be derived from naturally occurring or recombinant single- or double-stranded polynucleotides, or be chemically synthesized. Probes may also be labeled by nick translation, Klenow fill-in reaction, or other methods known in the art.

The design of probes with the appropriate size and sequence for preferential binding to target specific sequences as well as hybridization conditions for their use is well known in the art. See, e.g., Current Protocols In Molecular Biology, Volumes 1, units 2, 4, and 6, Frederick M. Ausubel at al. eds., 1995. Portions of polynucleotide sequences having at least about eight nucleotides, usually at least about 15 nucleotides, and fewer than about 6 kb, usually fewer than about 1.0 kb, from a polymorphic sequence are preferred as probes. Also contemplated are probes having a specific portion of a polymorphic sequence. Moreover, probes which are proximal to a polymorphic region may also be used in evaluating nucleic acid samples.

As noted above, a number of non-PCR based screening assays are contemplated in this invention. One procedure hybridizes a nucleic acid probe (or an analog such as a methyl phosphonate backbone replacing the normal phosphodiester), to the DNA target present at a low concentration. This probe may have an enzyme covalently linked to the probe, such that the covalent linkage does not interfere with the specificity of the hybridization. This enzyme-probe-conjugate target nucleic acid complex can then be isolated away from the free probe enzyme conjugate and a substrate is added for enzyme detection. Enzymatic activity is observed as a change in color development or luminescent output resulting in an increase in sensitivity. For an example relating to the preparation of oligodeoxynucleotide-alkaline phosphatase conjugates and their use as hybridization probes, see Jablonski et al., N.A.R., 14: 6115-6128, 1986. Two-step label amplification methodologies are known in the art. These assays work on the principle that a small ligand (such as digoxigenin, biotin, or the like) is attached to a nucleic acid probe capable of specifically binding an IMPDH2 gene region sequence. Allele specific probes are also contemplated within the scope of this example and exemplary allele specific probes include probes encompassing the predisposing polymorphisms of this patent application.

In one example, the small ligand attached to the nucleic acid probe is specifically recognized by an antibody-enzyme conjugate. In one embodiment of this example, digoxigenin is attached to the nucleic acid probe. Hybridization is detected by an anti-digoxigenin antibody conjugated to alkaline phosphatase conjugate. The alkaline phosphatase modifies a chemiluminescent substrate which can then be detected. For methods for labeling nucleic acid probes according to this embodiment see Martin et al., BioTechniques 9: 762-768, 1990. In a second example, the small ligand is recognized by a second ligand-enzyme conjugate that is capable of specifically complexing to the first ligand. A well known embodiment of this example is the biotin-avidin type of interaction. For methods for labeling nucleic acid probes and their use in biotin-avidin based assays see Nguyen et al., BioTechniques 13: 116-123, 1992. It is also contemplated within the scope of this invention that the nucleic acid probe assays of this invention will employ a combination of nucleic acid probes capable of detecting IMPDH2, IL 10 and MDR1 polymorphisms. Thus, in one example to detect the presence of polymorphisms in a cell sample, more than one probe complementary to the said genes is employed and in particular the number of different probes is alternatively two or three different nucleic acid probe sequences. The cocktail includes probes capable of binding to the allele-specific polymorphisms identified in populations of patients with alterations in this region. In this embodiment, any number of probes can be used, and will preferably include probes corresponding to the major polymorphisms in patients with acute renal transplant rejection.

Any one of the methods to detect polymorphisms described above can also be used for the optional detection of the IL 10 or MDR1 polymorphisms.

“Polynucleotide” and “nucleic acid” refer to single or double-stranded molecules which may be DNA, comprised of the nucleotide bases A, T, C and G, or RNA, comprised of the bases A, U (substitutes for T), C, and G. The polynucleotide may represent a coding strand or its complement. Polynucleotide molecules may be identical in sequence to the sequence which is naturally occurring or may include alternative codons which encode the same amino acid as that which is found in the naturally occurring sequence (See, Lewin “Genes V” Oxford University Press Chapter 7, pp. 171-174 (1994). Furthermore, polynucleotide molecules may include codons which represent conservative substitutions of amino acids as described. The polynucleotide may represent genomic DNA or cDNA. The nucleic acid may also be a synthetically generated DNA (e.g. the product of a PCR amplification).

“Specific hybridization,” as used herein, refers to the ability of a first nucleic acid to hybridize to a second nucleic acid in a manner such that the first nucleic acid does not hybridize to any nucleic acid other than to the second nucleic acid (e.g., when the first nucleic acid has a higher similarity to the second nucleic acid than to any other nucleic acid in a sample wherein the hybridization is to be performed). “Stringency conditions” for hybridization is a term of art which refers to the incubation and wash conditions, e.g., conditions of temperature and buffer concentration, which permit hybridization of a particular nucleic acid to a second nucleic acid; the first nucleic acid may be perfectly (i.e., 100%) complementary to the second, or the first and second may share some degree of complementarity which is less than perfect (e.g., 70%, 75%, 85%, 95%). For example, certain high stringency conditions can be used which distinguish perfectly complementary nucleic acids from those of less complementarity. “High stringency conditions”, “moderate stringency conditions” and “low stringency conditions” for nucleic acid hybridizations are explained on pages 2.10.1-2.10.16 and pages 6.3.1-6.3.6 in Current Protocols in Molecular Biology (Ausubel, F. M, et al., “Current Protocols in Molecular Biology”, John Wiley & Sons, (2001)), the entire teachings of which are incorporated by reference herein). The exact conditions which determine the stringency of hybridization depend not only on ionic strength (e.g., 0.2×SSC, 0.1×SSC), temperature (e.g., room temperature, 42° C., 68° C.) and the concentration of destabilizing agents such as formamide or denaturing agents such as SDS, but also on factors such as the length of the nucleic acid sequence, base composition, percent mismatch between hybridizing sequences and the frequency of occurrence of subsets of that sequence within other non-identical sequences. Thus, equivalent conditions can be determined by varying one or more of these parameters while maintaining a similar degree of identity or similarity between the two nucleic acid molecules. Typically, conditions are used such that sequences at least about 60%, at least about 70%, at least about 10%, at least about 90% or at least about 95% or more identical to each other remain hybridized to one another. By varying hybridization conditions from a level of stringency at which no hybridization occurs to a level at which hybridization is first observed, conditions which will allow a given sequence to hybridize (e.g., selectively) with the most similar sequences in the sample can be determined.

Exemplary conditions are described in Krause, M. H. and S. A. Aaronson, Methods in Enzymology 200:546-556 (1991), and in, Ausubel, et al. “Current Protocols in Molecular Biology”, John Wiley & Sons, (2001), which describes the determination of washing conditions for moderate or low stringency conditions. Washing is the step in which conditions are usually set so as to determine a minimum level of complementarity of the hybrids. Generally, starting from the lowest temperature at which only homologous hybridization occurs, each ° C. by which the final wash temperature is reduced (holding SSC concentration constant) allows an increase by 1% in the maximum extent of mismatching among the sequences that hybridize. Generally, doubling the concentration of SSC results in an increase in Tₘ of −17° C. Using these guidelines, the washing temperature can be determined empirically for high, moderate or low stringency, depending on the level of mismatch sought.

For example, a low stringency wash can comprise washing in a solution containing 0.2×SSC/0.1% SDS for 10 minutes at room temperature; a moderate stringency wash can comprise washing in a pre-warmed solution (42° C.) solution containing 0.2×SSC/0.1% SDS for 15 minutes at 42° C.; and a high stringency wash can comprise washing in pre-warmed (68° C.) solution containing 0.1×SSC/0.1% SDS for 15 minutes at 68° C. Furthermore, washes can be performed repeatedly or sequentially to obtain a desired result as known in the art. Equivalent conditions can be determined by varying one or more of the parameters given as an example, as known in the art, while maintaining a similar degree of identity or similarity between the target nucleic acid molecule and the primer or probe used.

The present invention also provides isolated nucleic acid molecules that contain a fragment or portion which includes a C at position 3757 of Seq ID No. 1 which includes position 3757 of Seq ID No. 1 and that hybridizes under highly stringent conditions to Seq ID No.1 wherein the T at position 3757 is replaced by a C, or the complement of said sequence. The nucleic acid fragments of the invention are at least about 15, preferably at least about 18, 20, 23 or 25 nucleotides, and can be 30, 40, 50, 100, 200 or more nucleotides in length. As a non-limiting example, one such fragment may be a nucleic acid comprising the sequence from position 3497 to 3938 of Seq ID No. 1, wherein nucleotide T at position 3757 is replaced by a C. Such fragments may also be probes or primers hereinbefore described.

In a related aspect, the nucleic acid fragments of the invention are used as probes or primers in assays such as those described herein. “Probes” or “primers” are oligonucleotides that hybridize in a base-specific manner to a complementary strand of nucleic acid molecules. Such probes and primers include polypeptide nucleic acids, as described in Nielsen et al., Science 254:1497-1500 (1991).

A probe or primer comprises a region of nucleotide sequence that hybridizes to at least about 15, for example about 20-25, and in certain embodiments about 40, 50 or 75, consecutive nucleotides of a nucleic acid molecule comprising a contiguous nucleotide sequence from Seq ID No. 1 wherein the T at position 3757 is replaced by a C. Optionally, such probes derived from Seq ID No. 5 or 6 can be used in addition to the probes or primers described above. The nucleic acid molecules of the invention such as those described above can be identified and isolated using standard molecular biology techniques and the sequence information provided herein. For example, nucleic acid molecules can be amplified and isolated by the polymerase chain reaction using synthetic oligonucleotide primers designed based on Seq ID No. 1, 5 or 6 or the complement of such a sequence. See generally PCR Technology: Principles and Applications for DNA Amplification (ed. H. A. Erlich, Freeman Press, NY, N.Y., 1992); PCR Protocols: A Guide to Methods and Applications (Eds. Innis et al., Academic Press, San Diego, Calif., 1990); Mattila et al., Nucl. Acids Res. 19: 4967 (1991); Eckert et al., PCR Methods and Applications 1:17 (1991); PCR (eds. McPherson et al., IRL Press, Oxford); and U.S. Pat. No. 4,683,202. The nucleic acid molecules can be amplified using cDNA, mRNA or genomic DNA as a template, cloned into an appropriate vector and characterized by DNA sequence analysis.

To carry out the methods for assessing the susceptibility to acute renal transplant rejection in a patient, the present invention also refers to a kit comprising at least one reagent for use in detecting the T3757C polymorphism in the IMPDH gene, instructions setting forth a procedure according to any of the methods for assessing the susceptibility to acute renal transplant rejection hereinbefore described, and a container for contents of the kit. In a preferred embodiment, the at least one reagent for use in detecting the T3757C polymorphism in the IMPDH gene comprises a nucleic acid capable of specifically hybridizing to the nucleic acid of Seq ID No.1; or the nucleic acid of Seq ID No. 1 wherein the nucleotide T at position 375 is replaced by an C. In another preferred embodiment, said at least one reagent for use in detecting the T3757C polymorphism in the IMPDH2 gene comprises nucleic acid primers which hybridize to Seq ID No. 1 under stringent conditions and which can be used for dideoxy sequencing of a portion of Seq ID No. 1 which includes position 3757. Such nucleic acid may be the nucleic acids of Seq ID No. 2, 3 and/or 4.

In a more preferred embodiment, the kit additionally comprises at least one reagent to detect the C3435T polymorphism in the MDR1 gene and/or the −592C>A polymorphism in the IL10 gene. In a most preferred embodiment, the kit comprises the nucleic acids of Seq ID No.s 2 to 4 and/or 7 to 9 and/or 10 to 11.

### EXAMPLES

### Example 1

### Cohort Description

The analysis was conducted in a cohort of 237 Caucasian transplant patients who consented to the pharmacogenetic study described herein, out of the 536 patients who participated in the CAESAR clinical study. CAESAR was a 12 month, open label, controlled multicenter prospective study designed to assess renal function, graft and patient survival and biopsy proven acute rejection (BPAR) in de novo primary renal allograft recipients.

The main aim was to minimize nephrotoxicity and improve long term renal function and graft survival without adversely affecting efficacy, by reducing or eliminating cyclosporine use.

Patients were randomized prior to transplantation into 1 of 3 groups. (1) daclizumab (dac), mycophenolate mofetil (MMF), corticosteroids (CS), low dose cyclosporine (CsA) followed by weaning (month 4) and withdrawal (month 6); (2) dac, MMF, CS, low dose CsA; (3) MMF, CS, standard dose CsA.

### Selection of Patients

The pharmacogenetic study protocol and the informed consent form were submitted for approval to the local ethical committees in the respective countries. All patients provided written informed consent for their blood sample to be used for genotyping. The sample could be withdrawn up to a month later, if the patients changed their mind.

All the samples were assigned new independent codes and a month after sample collection the link between the new and original codes was deleted. This was an added measure to ensure patient confidentiality; however, as a consequence it is not possible to retrieve genotype information based on the patient's name or number used in the original clinical trial. In approximately 15 years time, all blood and DNA samples will be destroyed.

### Preparation of Samples

Single blood samples (9 ml) were collected in EDTA tubes. These were frozen and stored between ±20 and ±70° C., before being sent to the Roche Central Sample Office (CSO) in Basel, Switzerland, where they were aliquoted into three tubes and assigned new, independent codes on barcode labels to assure patient anonymity. Two samples of blood (1 ml and 4 mls) were sent to the Roche Sample Repository (RSR) at Roche Center for Medical Genomics (RCMG) in Basel, Switzerland. The remaining 4 ml aliquot was stored at −80° C. in the CSO in Basel, Switzerland. All procedures performed on the samples at the RSR were done according to established standard operating procedures using GCP guidelines.

DNA was extracted from 200 μl of the whole blood using a silica gel-based extraction method (MagNA Pure LC DNA isolation KIT I, Roche Molecular Biochemicals). Samples were genotyped for two different single nucleotide polymorphisms (SNPs) using a combination of the amplification refractory mutation system (ARMS) that relies on 3′ terminal mismatches between the PCR primers and the template being amplified according to Newton et al., Nucleic Acids Res. (1989), 17(7), 2503-16 for SNPs in MDR1 and IL 10 and sequencing analysis for the SNP in IMPDH2.

Analysis of two point mutations in DNA was transformed by using the amplification refractory mutation system (ARMS, Nucleic Acids Res. (1989), 17(7), 2503-16) and using the kinetic thermal cycler (KTC) format of the polymerase chain reaction. This method allows discrimination of single nucleotide polymorphisms (SNP) in a single-tube without the use of fluorescent probes (Higuchi et al., Biotechnology (1993), 11, 1026-1030).

In the KTC format, the generation of double-stranded amplification product is monitored using a DNA intercalating dye and a thermal cycler which has a fluorescence-detecting CCD camera attached (ABI-GeneAmp 7900 Sequence Detection System). Fluorescence in each well of the PCR amplification plate is measured at each cycle of annealing and denaturation. The cycle at which the relative fluorescence reached a threshold of 0.5 using the SDS software from PE-Biosystems was defined as the Ct.

The amplification reactions were designed to be allele-specific, so that the amplification reaction was positive if the polymorphism was present and the amplification reaction was negative if the polymorphism was absent. For each bi-allelic polymorphism, one well of the amplification plate was set up to be specific for allele 1 and a second well was set up to be specific for allele 2. For each polymorphism to be detected, three primers were designed—two allele-specific primers and one common primer (Table 3). Reactions for allele 1 contained allele 1-specific primer and the common primer and reactions for allele 2 contained allele 2-specific primer and the common primer.

TABLE 1
list of oligonucleotide primers used for polymorphism detection

| | | | | Primer | |
|---|---|---|---|---|---|
| | Primer | | SEQ | concentration | Annealing |
| Marker | type | Nucleotide sequence | ID NO | (in μM) | temperature |
| IL 10 −592 C > A | AS1 | GTGACCCCGCCTGTC | 2 | 0.2 | 65 |
| | AS2 | GTGACCCCGCCTGTA | 3 | 0.4 | 65 |
| | common | ACTTTCCAGAGACTGGCTTCCTAC | 4 | 0.2 | 65 |
| MDR1 C3435T | AS1 | TCCTTTGCTGCCCTCACG | 7 | 0.2 | 60 |
| | AS2 | CTCCTTTGCTGCCCTCACA | 8 | 0.2 | 60 |
| | common | GGGTGGTGTCACAGGAAGAGA | 9 | 0.2 | 60 |
| IMPDH2 | F | CTTCAGGGCACAATCTTGCC | 10 | 0.4 | 61 |
| T3657C | R | CCTGGAACTAATGCCTAGGG | 11 | 0.4 | 61 |

The amplification conditions were as follows for IL 10 and MDR1:
50 mM Tris pH 8.0,
50 mM KCl,
3 mM MgCl2,
50 μm each of dATP, dCTP, and dGTP,
25 μm of TTP and 75 μm of dUTP,
4% DMSO,
2 μM ROX (Carboxy-Rhodamine)
0.1× SyBr Green (Molecular Probes, Eugene, Oreg.),
2% glycerol,
uracil N-glycosylase (UNG,2 units),
Delta Z05 Gold DNA polymerase (3 units)
and primers in an 20 μl volume for each well.

The concentration of the primers used for each assay is listed in Table 1. 5 ng of DNA in 5 μl volume was then added to each well. To reduce the possibility of contamination by pre-existing amplification product, the assay procedure included the incorporation of dUTP into the amplification product and an incubation step for UNG degradation of pre-existing U-containing products (Longo et al, Gene (1990), 93, 125-128). Amplification reactions were prepared using an aliquoting robot (Tecan) in 384-well amplification plates identified by barcode labels generated by the labpratory management database. Parameters for procedures performed by the robot were set to minimize the possibility of cross-contamination. For each plate of 81 samples, 5 samples were run in duplicate and the duplicate results were analyzed to determine that they matched. The thermal cycling conditions were as follows: 2 minutes at 50° C. for UNG degradation of any previously contaminating PCR products, 12 minutes at 95° C. for Delta Z05 Gold DNA polymerase activation, 45 cycles of denaturation at 95° C. and annealing at the annealing temperature indicated in Table 1, followed by a dissociation step of 1 minute at 1 degree increments from 60° to 95° C. The amplification reactions were run in ABI GeneAmp 7900 Sequence Detection Systems instruments. The first derivatives of the dissociation curves were produced by the SDS software and examined as needed to confirm that the fluorescence in a given reaction was due to amplification of a specific product with a well-defined dissociation peak rather than non-specific primer-dimer. Product differentiation was done by Analysis of DNA Melting Curves during PCR following the method of K. M. Ririe et al., Anal. Biochem. (1997), 245, 154-160.

The Cycle threshold Ct of each amplification reaction was determined and the difference between the Ct for allele 1 and allele 2 (delta Ct) was used as the assay result. Samples with delta Cts between −3.0 and 3.0 were considered heterozygous (A1/A2). Samples with delta Cts below −3.0 were considered homozygous for A1 (A1/A1); samples with delta Cts above 3.0 were considered homozygous for A2 (A2/A2). In most cases, the delta Ct differences between the three groups of genotypes were well-defined and samples with Ct values close to 3.0 were re-tested as discrepants. Each assay was run on a panel of 47 cell line DNAs to identify cell lines with the appropriate genotypes for use as controls on each assay plate (A1/A1, A1/A2, and A2/A2). The cell line DNA was obtained from the Corriel Institute and was extracted using the Qiagen extraction kits (QiaAmp DNA Blood kits, Valencia, Calif.). The genotypes of the cell line DNAs were confirmed by DNA sequencing. Three cell line DNAs (A1/A1, A1/A2, and A2/A2) were run as controls on each plate of clinical trial samples and used to determine the between-plate variability. The Ct values obtained for the control cell lines were analysed to determine the cut-off for the delta Ct values obtained for the clinical trial samples. A data file containing the Ct values for each well was generated by the SDS software and entered into the experiment management database. A data file with the final genotypes identified by the independent code was extracted from the database and matched to the clinical data also identified by the independent code for the statistical analysis.

For IMPDH2 single nucleotide polymorphism (SNP) genotyping was done by double-stranded DNA sequencing using an ABI capillary sequencer and Big Dye chemistry (ABI). The primers used to amplify the exon7/intron7 boundary are shown in table 1 and were also used as sequencing primers. Publicly available genomic sequences were used as references for primer design. All polymorphisms were targeted with these pairs-of-primer sets:

Primer IL 10-592C>A AS1 corresponds to positions 668 to 682 in the promoter sequence of IL10 as defined by the positions in SEQ ID NO:1.
Primer IL 10-592C>A AS2 corresponds to positions 668 to 682 in the promoter sequence of IL10 as defined by the positions in SEQ ID NO:1.
Primer IL 10-592C>A Common corresponds to the complementary strand and hybridizes to positions 708 to 685 in the promoter sequence of IL10 as defined by the positions in SEQ ID NO:1.
Primer MORI C3435T AS1 corresponds to the complementary strand and hybridizes to positions 193 to 176 in exon 26 of MDR1 as defined by the positions in SEQ ID NO:5.
Primer MDR1° C.3435T AS2 corresponds to the complementary strand and hybridizes to positions 194 to in exon 26 of MDR1 as defined by the positions in SEQ ID NO:5.
Primer MDR1 C3435T Common corresponds to positions 154 to in exon 26 of MDR1 is as defined by the positions in SEQ ID NO:5.
Primer IMPDH2 T3757C F corresponds to the complementary strand and hybridizes to positions 3938 to 3919 in intron 6 of IMPDH2 as defined by the positions in SEQ ID NO:6.
Primer IMPDH2 T3757C R corresponds to positions 3497 to 3516 in intron 8 of IMPDH2 as defined by the positions in SEQ ID NO:6.

For IMPDH2 PCR twenty nanograms of genomic DNA were PCR-amplified in 20 ul reactions using a MJ research Tetrad PCR machine.

For the amplification of the IMPDH2 fragment conditions were as follows:

### 50 mM Tris pH 8.3,

### 10 mM KCl,

### 1.5 mM MgCl2,

0.2 mM of each dNTP,
0.4 μM of each primer and

### 0.6 U AmpliTaq Gold Polymerase.

The thermocycling protocol consisted of an initial incubation of 95° C. for 15 min. followed by 35 cycles of 94° C. for 1 min., 61° C. for 30 sec., 72° C. for 1 min., and one final extension step of 72° C. for 5 min.

Afterwards, PCR amplification fragments were purified using Millipore PCR Cleanup plates on a Tecan biorobot. Cycle sequencing was performed on a MJ Tetrad PCR machine using ABI Big Dye terminator chemistry according to the manufacturer's instruction. After sequencing, the polymorphism analyses were done using Polyphred software (licenced from University of Washington).

### Example 2

### Univariate Snp Analysis

The analysis was first conducted for each polymorphism. The association between occurrence of BPAR (Biopsy Proven Acute Rejection) and each polymorphism was assessed after adjusting for treatment group gender and age (coded as a two categories variable, <=50 y, >50 y), using a logistic regression model.

The hypothesis of absence of association between the genotypes for each polymorphism and the occurrence of BPAR was tested using a genotypic test (Sasieni 1997). This test is an independence test between the genotypes and the occurrence of BPAR which, in the case of MDR1 C3435T and IL10-592C>A, follows a chi-square with 2 degrees of freedom. For IMPDH2 T3757C, the C/C homozygote group was pooled with the C/T genotype because it contained only 2 individuals. Therefore, the genotypic test has only 1 degree of freedom df. The type 1 error of the genotypic test was set to 0.05 and no adjustments for multiple comparisons were made.

For MDR1 C3435T polymorphism, an allelic trend test (Sasieni, 1997) was also performed to test for the absence of association between the alleles of this polymorphism and the occurrence of BPAR. The allelic trend test is testing the independence between the presence of 1 allele and the occurrence of BPAR. The corresponding odds ratio, as well as its 95% confidence interval was computed. (see table below).

For IMPDH2 T3757C, the corresponding odds ratio ({C/C U C/T} versus T/T) was computed with its relative 95% confidence interval.

For IL 10-592C>A, a recessive coding test was also computed, by pooling the C/C and the A/C genotype together, since the odds of developing BPAR in patients with these genotypes were shown to be very similar by the genotypic test. The corresponding chi-square with 1 df was computed, as well as the odds ratio of BPAR associated with A/A genotypes compared to the A/C or C/C genotypes.

As shown in table 2, all 3 polymorphisms were significant using the genotyping test.

The presence of the IMPDH2 C allele increased the odds of developing BPAR by almost 3 times (odds ratio (OR) 2.99, 95% CI:1.27-6.99; p=0.012); the presence of the MDR1 T allele almost doubled the odds (OR 1.98, 95% CI:1.24-3.14; p=0.004); the presence of the IL 10 A allele was associated with nearly 5-fold higher odds (OR 4.76, 95% CI:1.59-14.26; p=0.005). No significant interactions between treatment group and genotype were detected for these 3 polymorphisms.

TABLE 2
Univariate pharmacogenetic analysis results

| | | Genotypes (%) | | |
|---|---|---|---|---|
| MDR1 C3435T snp | n | T/T | C/T | C/C |
| BPAR event | 64 | 20 (37.7) | 35 (29.9) | 9 (16.1) |
| No BPAR event | 162 | 33 (62.3) | 82 (70.1) | 47 (83.9) |

Genotypic test: χ² = 6.60, df = 2, p = 0.037
Allelic trend test: χ² = 8.32, df = 1, p = 0.004
Allelic OR T vs. C = 1.98 [1.24; 3.14]

| | | Genotypes (%) | |
|---|---|---|---|
| IMPDH2 T3757C snp | n | T/C U C/C | TT |
| BPAR event | 61 | 13 (46.4) | 48 (24.9) |
| No BPAR event | 160 | 15 (53.6) | 145 (75.1) |

Genotypic test: χ² = 5.69, df = 1, p = 0.017
OR [T/C U C/C] vs. TT = 2.99 [1.27; 6.99]

| | | Genotypes (%) | | |
|---|---|---|---|---|
| IL10 −592 C > A snp | n | A/A | A/C | C/C |
| BPAR event | 60 | 10 (55.6) | 18 (25.0) | 32 (25.2) |
| No BPAR event | 157 | 8 (44.4) | 54 (75.0) | 95 (74.8) |

Genotypic test: χ² = 7.64, df = 2, p = 0.022
Recessive coding test (A/A vs {A/C U C/C}),
χ²= 7.87, df = 1, p = 0.005
OR A/A vs {A/C U OC} = 4.76 [159; 14.26]
Legend: n: sample size per group; OR: odds ratio (Odds of Event; Odds of no event) [95% confidence interval of the OR]; Genotypic test, allelic trend test, recessive coding test: see text; df: degrees of freedom; p: p-value; BPAR: Biopsy Proven Acute Rejection; df: degree of freedom.

### Example 3

### Multivariate Snp Analysis

The three significant polymorphisms (see table 2) were combined in a logistic regression model to assess their significance adjusted on the effects on the two others. The three polymorphisms were added using the coding described above.

As previously described, the analysis was performed taking into account treatment group, gender and age. The results are presented in table 3.

When included in the same multivariate model, all 3 polymorphisms were significant at the 5% type I error level.

TABLE 3
Multivariate pharmacogenetic results

| | | | p-value in | Odds Ratio (95% |
|---|---|---|---|---|
| polymorphism | Wald χ² | df | final model | confidence interval) |
| MDR1, C3435T | 8.07 | 1 | 0.005 | 2.07 [1.25; 3.49] |
| IL10 −592 C > A | 6.45 | 1 | 0.011 | 4.60 [1.42; 14.94] |
| IMPDH2, T3757C | 4.90 | 1 | 0.027 | 2.84 [1.13; 7.14] |

1. A method for assessing the susceptibility to acute renal transplant rejection in a patient comprising
a) Isolating a nucleic acid from a sample that has been removed from the patient and
b) Detecting the nucleotide present at position 3757 of SEQ ID NO. 1, wherein the presence of an C at this position is indicative of acute renal transplant rejection.

2. The method of claim 1, further comprises detecting an additional one or more other marker for the prediction of acute renal transplant rejection wherein said one or more other marker(s) is the nucleotide present at position 176 of SEQ ID NO. 5 and/or the nucleotide present at position 682 of SEQ ID NO.6.

3. The method of claim 2, wherein said sample is whole blood.

4. The method of claim 2, wherein said detecting is achieved by dideoxy sequencing and/or allele-specific quantitative PCR.

5. The method of claim 4, wherein said allele-specific PCR is performed using the allele-specific primers of SEQ ID No. 2 and SEQ ID NO. 3 and the common primer of SEQ ID NO. 4 and/or the allele-specific primers of SEQ ID NO. 7 and SEQ ID NO. 8 and the common primer of SEQ ID NO. 9 and/or said dideoxy sequencing is performed with the allele-specific primers of SEQ ID NO. 10. and SEQ ID NO. 11.

6. A kit for assessing the susceptibility to acute renal transplant rejection in a patient comprising at least one reagent for use in detecting the T3757C polymorphism in the IMPDH2 gene, instructions setting forth a procedure according to the method of claim 2, and a container for contents of the kit.

7. The kit of claim 6, wherein the at least one reagent for use in detecting T3757C polymorphism in the IMPDH2 gene comprises a nucleic acid capable of specifically hybridizing to the nucleic acid of SEQ ID NO. 1; or the nucleic acid of SEQ ID NO. 1 wherein the nucleotide T at position 3757 is replaced by a C.

8. The kit of claim 7, additionally comprising at least one reagent to detect the C3435T polymorphism in the MDR1 gene and/or the −592C>A polymorphism in the IL10 gene.

9. (canceled)


SEQUENCE LISTING (11 sequences)

SEQ ID NO: 1 - DNA, 6193 bp, Homo sapiens
ggccaagagaaaccagccaggaaaaaccagacagactttcacactaaagaagaggcctccatttttttttttcttttttttattggtgtagttacgaagcctttcaggctgcttctgtttaaaatataaaagaaaactttgccccctttgcatcttcataaacctgctgcggcagactcctcagccgatggtggctctgggtttccttgagtgtcatatgtcctagaaagttgctggctgactcttttttgtctggggcctggggaaagggcttggactgtgaaaagaaatgtggcccctttccatcttcaagagagatggaattaatgatggatggaccctggagggaatctccccagccgacttccactgggctgacagactttgctgaccacaggggaacgatgttcttttctttcttcatgatcagacataaacttagcatcttaatggaagaaaaatgaggggaacttcaattatgatttattaaagacaatttctattacaccctcctttatgacaagtgacattttagatgtaaaagtaaaaactttaccatgccttttttttttttgttggcctaacattgaggccttaaaacctgaggctcctgtgcctgatggaattcttgtaacatacacttgtgtatcatataaagataccactctgtttctcttatgtattcttactctagttgtttattaagaatgacaagcacgtcttttcaacatgttagtgaacaacgtctctttattttggggaggagcccggtgggacagtagaagtaaacccttgcctgttaattgaactggccgaggtcctgcggggagtgtggggtgggacgtgaagtggcagctcctcagtgacaaggccactatgtgctatacgcatgcgctgtttcttcagcgccagctccgcccccgccgcagcgaggcgggtaggttccgcccgcgcgcactacgccctgacgtcagcgtcgcgcgcagcagtgacgaaatcggctggtttatattggcgcggcccagacggcagaggtctctgcggcgcggtcctcggagacacgcggcggtgtcctgtgttggccatggccgactacctgattagtgggggcacgtcctacgtgccagacgacggactcacagcacagcagctcttcaactgcggagacggcctcacctacaagtgcgggcctatggggctcacctgggcgaagagaggggcggcacctggggaaaaggccagatgggggcttccgagagccgccttgggcgtgggcgtgggggacaggtttcgggaattggccacccacctccggggctcactttgagacatctggagcggtgcttggggtacggagaagcatctcagtagtgcttggaatctcaggccggagattccatgctccagacacatggaattgtcagggtgccaggagcactgatgacagctcggtggcagaaaaggtcccctgtacccatttccaccttgtgctgtgcctgtgacacacagactctgtcagtgccactctctgctgacatgcgttacgggttgattggtacatggggatggagtctcatgctgccctaagctgagagctttactcccaattcactcccttccctcgcagtgactttctcattctccctgggtacatcgacttcactgcagaccaggtggtgagtatgatcaggaattgggtcctgaacataaggtgagctaaggtgctactatcacctggtctctgggctaactcaccagattgggcttgggggtggattttataggatcatgttgcttgttagcatgcaggtcatacaaaagtgccttttgtgggggactcagtaacaccttgaggtgtaagggatgctttcccacactggttgacgtttgtctcctcaggacctgacttctgctctgaccaagaaaatcactcttaagaccccactggtttcctctcccatggacacagtcacagaggctgggatggccatagcaatggcggtgagccccatggggtgtgggggaaggagcaaggactccatcgcctttccccaaaggcattcagtcctgcttcttgtcacttagtagtagtctctttttatcctgtagcttacaggcggtattggcttcatccaccacaactgtacacctgaattccaggccaatgaagttcggaaagtgaaggtcagaagggcaacgatcattagcaagcgctcctgggaattgcactgaggtggggtggggtgggagtaggggtttattctaatttagtattctttcttcccaccatggggttcagttactgagaagaccctgagattctgtttcttaaagcagcagcaatagaccaggtgtacagtgcctccagcctacccatgtctctaagatgtgttggtgtgatttggtcttgtggcactgccaaagggatcgataagcagagaccccatgcttcagatcaagagcctgatgaaagtagttcaaagatgcgatgccctttctcaccatccctttccagaaatatgaacagggattcatcacagaccctgtggtcctcagccccaaggatcgcgtgcgggatgtttttgaggccaaggcccggcatggtttctgcggtatcccaatcacagacacaggccggatggggagccgcttggtgggcatcatctcctccagggacattgattttctcaaagaggaggaacatgactgtttcttggaagaggtgggtgccactggcagagtagatccaagctctagcagcccaggttgaagacaaggggcttctgttgtctaagacatctctgatagcctcttctctctgggaaaggagtgtcaaaccaagtttctgactcctttattgggttcctgcatccttcccctccagaggctttcaaactgcccaaatgcttgctagtattcatgctttgttcttttaaacccctaaggtaggggcagtgcttgaggtctgccctgattttcttgccattgttcctgtgttgtcctccttattccatagtgtaagagggtgctccctgtgccatgttgtcctttctactcatccctcactcaataccatactgctaagaattattgggatcccttgaggaagggtgttttgggtgtgaatgtatgaacgtggtggtccatagaacttcacccctgtaatctcagtactttgggagacttgaggcaggcaaatcatgaggttaggagtttgagaccagcctggccaacatggtgaaaccccgtctctactaaaaatacaaaaaattagttgggtgtggtggcgcgtgcctctgagctcagctacttgggaaggaaagaaaagagttaactggtcatagtcgatgactggcccttcttcacatctcacctcccacgtagataatgacaaagagggaagacttggtggtagcccctgcaggcatcacactgaaggaggcaaatgaaattctgcagcgcagcaagaagggtaagtcctagagctgggaaagggcctggaactaatgcctagggtcctgattcatgtcctgccctgaccacaggaaagttgcccattgtaaatgaagatgatgagcttgtggccatcattgcccggacagacctgaagaagaatcgggactacccactagcctccaaagatgccaagaaacagctgctgtgtggggcagccattggcactcatgaggatgacaagtataggctggacttgctcgcccaggctggtgtggatgtagtggttttgggtgagcctgctacacaggtgggttggggcaataggggcagctgccacatgacagtctgagacttacttcttgtcctagactcttcccagggaaattccatcttccagatcaatatgatcaagtacatcaaagacaaataccctaatctccaagtcattggaggcaatggtaaggcaagattgtgccctgaaggatgtgtggtgggagtgggtaagctgggctcccaccttaaccttcacatgagcattttcctttccttcaccatagtggtcactgctgcccaggccaagaacctcattgatgcaggtgtggatgccctgcgggtgggcatgggaagtggctccatctgcattacgcaggaaggtaagaatatactttgatagggcccacagagaccccagtttcaggccccacataccttggaaccaagcactcttatggggacaatagagccctccaataggggcaaccctggggccaaattggcctcttggatgggtgacatggctggaagcaagacaaggtcttgtcatccatcacctgccactcccttgcctagcctggccttcttgctcctgctctgcaccttctctaaactctggtctgtttgttttatttagtggctcccaagatccctccagacataaagtcccacagccccaaatgcccttctactgtcacgggttgctatagtaagtccagcccggcccactggcccggcccagctgcccactgcccggctgcttggttgacgtagctgtagctcccggggtttgtggtgctgatgggtgggcaaggccaactgatacacagaccatggtttgggggtggtgctcaagaacatagctccctcttccttctggggtgctgatagggcaggaccttcttccctaggcaggaccttttctgcccacatcttctgcagcaggcatagttcagggaccaactcctttgtgtggtccttgctctcccacacaaccactgcctgctccacacaaccacagcctgctctcctacacaaccactgcccttatcttgcctgagctgcccaccattcagggaggaagggctgggcctgacctcagtgcctctggggactaactgcacggtgactcctgccccatctgataccagctggacggggctttcccggctgctaagactgcatgtgcctgaggctgccctgggcactgcacacatgcacgtgcacatgtgggggtgagtagaggtcagcacagctgtttgtactatttaaggcagatgtggcagcagccatcccagacacgtctgttcctcagccaagctgcagtcctgatgcagatgtagccaaaagctcctgggtcctaaatatggccacagggtccactgcccgtccccattaaccctatccacccatgtgttcctccatctcaacagtgctggcctgtgggcggccccaagcaacagcagtgtacaaggtgtcagagtatgcacggcgctttggtgttccggtcattgctgatggaggaatccaaaatgtgggtcatattgcgaaagccttggcccttggggcctccacagtcatgatgggctctctcctggctgccaccactgaggcccctggtgaatacttcttttccgatgggatccggctaaagaaatatcgcggtatgggttctctcgatgccatggacaagcacctcagcagccagaacagatatttcaggtgggacaggcaagccagttaccccaccctaatcctgcactgacagtctcatctctgctgtgaccttgcccgtgtctctgcctctccctgcagtgaagctgacaaaatcaaagtggcccagggagtgtctggtgctgtgcaggacaaagggtcaatccacaaatttgtcccttacctgattgctggcatccaacactcatgccaggacattggtgccaagagcttgacccaagtccggtgagcttggggagctgggacatgggtagaggggctggtccagggccagctacccacatttgacctctgcccttcttcagagccatgatgtactctggggagcttaagtttgagaagagaacgtcctcagcccaggtggaaggtggcgtccatagcctccattcgtaagtcaccctgtccttggtggggcctctgccatacctcatgcctcctttctcctgccctcacctcacaggtggtccttctgcctgcaggtatgagaagcggcttttctgaaaagggatccagcacacctcctcggtttttttttcaataaaagtttagaaagaaaaaagtgatgcctgatctttcaacagacaggtggggctctgtagctgccactaccacagatgcacacaaaaacagcaccctcatttccagggggagcctcaggccccgagataaatgtgctccatggacctggaagcgggtagttcaggtccaaaaagttcctctagctctcaca

SEQ ID NO: 2 - DNA, 15 bp, Artificial Sequence
  Description of Artificial Sequence Synthetic primer
gtgaccccgcctgtc

SEQ ID NO: 3 - DNA, 15 bp, Artificial Sequence
  Description of Artificial Sequence Synthetic primer
gtgaccccgcctgta

SEQ ID NO: 4 - DNA, 24 bp, Artificial Sequence
  Description of Artificial Sequence Synthetic primer
actttccagagactggcttcctac

SEQ ID NO: 5 - DNA, 247 bp, Homo sapiens
gatctgtgaactcttgttttcagctgcttgatggcaaagaaataaagcgactgaatgttcagtggctccgagcacacctgggcatcgtgtcccaggagcccatcctgtttgactgcagcattgctgagaacattgcctatggagacaacagccgggtggtgtcacaggaagagatcgtgagggcagcaaaggaggccaacatacatgccttcatcgagtcactgcctaatgtaagtctctcttcaaa

SEQ ID NO: 6 - DNA, 1327 bp, Homo sapiens
gatccccagagactttccagatatctgaagaagtcctgatgtcactgccccggtccttccccaggtagagcaacactcctcgtcgcaacccaactggctccccttaccttctacacacacacacacacacacacacacacacacacacacacacacaaatccaagacaacactactaaggcttctttgggagggggaagtagggataggtaagaggaaagtaagggacctcctatccagcctccatggaatcctgacttcttttccttgttatttcaacttcttccaccccatcttttaaactttagactccagccacagaagcttacaactaaaagaaactctaaggccaatttaatccaaggtttcattctatgtgctggagatggtgtacagtagggtgaggaaaccaaattctcagttggcactggtgtacccttgtacaggtgatgtaacatctctgtgcctcagtttgctcactataaaatagagacggtaggggtcatggtgagcactacctgactagcatataagaagctttcagcaagtgcagactactcttacccacttcccccaagcacagttggggtgggggacagctgaagaggtggaaacatgtgcctgagaatcctaatgaaatcggggtaaaggagcctggaacacatcctgtgaccccgcctgtcctgtaggaagccagtctctggaaagtaaaatggaagggctgcttgggaactttgaggatatttagcccaccccctcatttttacttggggaaactaaggcccagagacctaaggtgactgcctaagttagcaaggagaagtcttgggtattcatcccaggttggggggacccaattatttctcaatcccattgtattctggaatgggcaatttgtccacgtcactgtgacctaggaacacgcgaatgagaacccacagctgagggcctctgcgcacagaacagctgttctccccaggaaatcaactttttttaattgagaagctaaaaaattattctaagagaggtagcccatcctaaaaatagctgtaatgcagaagttcatgttcaaccaatcatttttgcttacgatgcaaaaattgaaaactaagtttattagagaggttagagaaggaggagctctaagcagaaaaaatcctgtgccgggaaaccttgattgtggctttttaatgaatgaagaggcctccctgagcttacaatataaaagggggacagagaggtgaaggtctacacatcaggggcttgctcttgcaaaaccaaaccacaagacagacttgcaaaagaaggcatgcacagctcagcactgc

SEQ ID NO: 7 - DNA, 18 bp, Artificial Sequence
  Description of Artificial Sequence Synthetic primer
tcctttgctgccctcacg

SEQ ID NO: 8 - DNA, 19 bp, Artificial Sequence
  Description of Artificial Sequence Synthetic primer
ctcctttgctgccctcaca

SEQ ID NO: 9 - DNA, 21 bp, Artificial Sequence
  Description of Artificial Sequence Synthetic primer
gggtggtgtcacaggaagaga

SEQ ID NO: 10 - DNA, 20 bp, Artificial Sequence
  Description of Artificial Sequence Synthetic primer
cttcagggcacaatcttgcc

SEQ ID NO: 11 - DNA, 20 bp, Artificial Sequence
  Description of Artificial Sequence Synthetic primer
cctggaactaatgcctaggg